(19) United States
(12) Patent Application Publication
(10) Pub. No.: US 2015/0087009 A1
(43) Pub. Date: Mar. 26, 2015

(54) LIVE CELL VIABILITY MODIFICATION SYSTEM AND METHOD

(71) Applicant: HOWARD LETOVSKY, WILLITS, CA (US)

(72) Inventor: HOWARD LETOVSKY, WILLITS, CA (US)

(21) Appl. No.: 14/558,967

(22) Filed: Dec. 3, 2014

Related U.S. Application Data
(63) Continuation-in-part of application No. 13/868,330, filed on Apr. 23, 2013, now Pat. No. 8,927,264.

(51) Int. Cl. C12N 13/00; C12M 1/42

(52) U.S. Cl. CPC C12N 13/00; C12M 35/02
USPC 435/29; 435/283.1; 435/287.1; 435/173.8

(57) ABSTRACT

An apparatus, system, and method are provided for selectively altering the viability characteristics of a live cell. In some embodiments, an electrical signal acquisition array, electrically coupled to a high gain amplifier, is used in conjunction with an analog to digital converter and a computer to record natural electrical signals that a cell type may use to communicate with its neighbors, the immune system, and the organism in which it is operating, as well as the signals and waveforms that define its particular natural operational electrical signature. Specific parts of the recorded electrical signals that are determined to be relevant to specific behavior patterns are extracted from the recordings as discreet waveforms, loaded into an arbitrary waveform generator, and played back through either an electromagnetic radiating transducer or an electrical signal output array to apply electromagnetic energy to the cells to alter the electrical characteristics of a live cell.

### RELATED APPLICATION

The application is a continuation-in-part of and claims the benefit of the filing date pursuant to 35 U.S.C. §120 of U.S. patent application Ser. No. 13/868,330, for an ELECTRO MEDICAL TOOL SYSTEM, filed Apr. 23, 2013.

### FIELD OF THE INVENTION

The present invention may relate to methods and apparatus intended to accelerate the healing rates of hard and soft human and/or animal tissues and promote regeneration of damaged organs using electrical and electromagnetic stimulation. This present invention may also relate to devices, systems, and methods that may sense electrical and/or molecular characteristics of at least one live cell, and more particularly, this invention may relate to devices, systems, and methods that may induce selective electrical changes in at least one live cell, wherein said selected electrical changes may catalyze molecular and/or chemical changes within the at least one live cell.

### BACKGROUND OF THE INVENTION

In every arena of medical practice, the healing of tissues is the primary problem that must be dealt with. Trauma induced contusions; abrasions, organ failures, and bone damage are medical issues dealt with by the millions daily. Typical medical approaches to trauma include stitches, bandages, casts, as well as simple and complex mechanical restructuring, then letting the body takes its natural healing course. Disease is rampant in current society as evidenced by the flood of “pills” offered on television and pharmacy shelves. The bulk of these products rarely heal the tissues themselves, but seek to neutralize the symptoms resulting in side effects that are often worse than the disease itself. Non-invasive tissue regeneration tools that have no side effects are needed.

Key prior art patents that may relate to the present invention may be presented herein with summaries of their abstracts.

A Yoshida et al. U.S. Pat. No. 5,922,209 may describe a process for deactivating or destroying microorganisms by applying electrical energy to a microorganism through a liquid, gas or solid having electrical energy to cause an increase in an electric charge in excess of the limit of intracellular and extracellular electrostatic capacity possessed by the microorganism, which in turn results in an irreversible change in the microorganism cells and/or explosively destroys the border membrane of the microorganism cells.

Chang's U.S. Pat. No. 5,304,486 may disclose a method of and apparatus for cell portion and cell fusion using radiofrequency electrical pulses. The method may be used to fuse or porate a variety of cells comprising animal cells, human cells, plant cells, protoplasts, erythrocyte ghosts, liposomes, vesicles, bacteria and yeasts. The method may also be used to produce new biological species, to make hybridoma cells which produce animal or human monoclonal antibodies and to insert therapeutic genes into human cells which can be transplanted back into the human body to cure genetic diseases.

Saban, et al. U.S. Pat. No. 6,790,341 may provide microband electrode array sensors for detecting the presence and measuring the concentration of analytes in a sample. The microband electrodes of the invention have both a width and thickness of microscopic dimensions. Preferably the width and thickness of the microband electrodes are less than the diffusion length of the analyte(s) of interest. The electrodes are separated by a gap insulating material that is large enough that the diffusion layers of the electrodes do not overlap such that there is no interference and the currents at the electrodes are additive.

Edwards, et al. U.S. Pat. No. 5,472,441 may disclose a device for treating body tissues containing cancerous cells or non-malignant tumors with RF ablation, alone or in combination with systemic or localized chemotherapy.

Harris, et al. U.S. Pat. No. 6,400,487 may teach methods and apparatus for screening large numbers of chemical compounds and performing a wide variety of fluorescent assays, comprising live cell assays. The methods utilize a laser line scan confocal microscope with high speed, high resolution and multi-wavelength capabilities and real time data-processing.

Chang's U.S. Pat. No. 8,278,629 may disclose live-cell observation equipment for a non light-transmitting microscope to study temperature-dependent events and method thereof.

Hofmann's U.S. Pat. No. 4,561,961 may describe a cooled microscope slide and electrode apparatus for use in live cell fusion system employing tubular electrodes so fluid may be pumped through the electrodes to dissipate heat to enhance the yield of viable hybrids. An alternate embodiment sandwiches a gasket and parallel tubular electrodes between glass slides to permit cell fusion in a closed sterile environment.

This inventor's own Letovsky U.S. Pat. No. 6,825,792 may disclose a frequency based missile detection and neutralization system that uses some similar components and creates some similar effects in non-organic compounds as the present invention does in organic compounds. This inventor's published patent application Letovsky 20110001064—as well as the parent patent from which it is a divisional—discloses aspects related to the present invention without comprising the camera image data to waveform generator feedback loop, the variable color source, direct contact electrical to cell system, and non contact electromagnetic frequency application components specification provided herein which are necessary to make the present invention function as intended.

In addition, the bioelectromagnetics and the therapeutic electro-medical tool industries have been evolving for over a hundred and fifty years in many parts of the world. However, the western medical mainstream is just starting to embrace these industries as providing effective alternatives to drug therapies. Electrical energy used in medical applications is applied to the body by physical contact with conducting electrodes or is radiatively coupled through a transducer that may be a coil, a waveguide, a light source, or other electromagnetic energy emitter. Most of the available tools use electrical signals drawn from effects observed over decades of trial and error.

There may be prior art related to sensing the electrical characteristics of live cells. The primary tools in this area are patch clamps, electrical impedance spectroscopy systems, and multi-electrode array probes and head stages. Patch clamps look for specific current information in response to electrical and chemical stimuli, electrical impedance spectroscopy looks at cellular impedance changes in response to said stimuli, and multi-electrode arrays are intended to provide data on neural responses to direct current electrical stimuli.

There may be also prior art related to sensing the molecular characteristics of live cells. The primary tools in this area are spectroscopy systems that are wavelength specific such as Raman, infrared absorption, and x-ray spectrometers.

Radiation from the sun, space, and the atmosphere, as well as brainwaves, bioelectric signals, water, and food chemistry drive living cell metabolic processes. When an organism's metabolism is out of balance with its baseline genetic programming, disease is the result. The electro medical tool industry uses electrical and electromagnetic frequencies from several hertz to light waves to assist in this metabolic rebalancing. The industry is now into its second century of evolution having begun largely in Eastern Europe.

Russian documentation detailing electrical physiotherapies goes from present day back to the mid 1800s, and incorporates sound, ultrasound, radio frequencies and specific light frequencies. Japanese carbon arc light healing tools date back to the second world war—healing radiation burn victims after atomic bombs were dropped on Hiroshima and Nagasaki. These highly specific carbon compounds create intense light outputs designed to reproduce specific combinations of light wave frequencies to expedite the body's natural healing processes. Clinical documentation of the effectiveness of this technology is very broad.

Scientists at the University of Alberta in Canada have successfully regenerated teeth from the root up—by the application of specifically configured 1.5 megahertz pulses. Electrical bone growth stimulation is now common throughout the world using specifically configured frequencies at 76.4 hertz, 40 kilohertz, and 1.5 megahertz to increase the speed of bone growth after a fracture or surgery.

Therapeutic “cold” lasers have been proven to increase cell metabolism, increase collagen synthesis for increased healing of soft tissues, increase osteoblast production for increased healing of bone, increase circulation through increased formation of new capillaries by release of growth factors, increase T-cell production for increased immune function, increase production of neurotransmitters such as endorphins, serotonin, ACTH, etc., and increase chronic pain threshold through decreased C-fiber activity.

Cancer cure rates with chemotherapy and radiation—though increasing significantly in certain types of cancers—are still lacking in long term cancer cell elimination after decades of research and billions of dollars spent. Electro therapy tools in global research centers are starting to gain traction as being potentially useful for targeting cancer cells while leaving the immune system and neighboring healthy cells undamaged.

In the U.S. (United States), electro medical therapy home use products are now showing up everywhere—mostly “copy catting” each other with a very small number of frequencies used in therapeutic ultrasound, therapeutic lasers, TENS machines, galvanic skin stimulators, frequency specific microcurrent generators, etc. Often, beneficial results of these tools are “hit or miss” with users—yet scores of these product offerings in the market. In general the electro therapy tools available to the public are limited in their accuracy and effectiveness due to a lack of direct testing on live cells both inside and outside living bodies due to a lack of combined observation, analysis, and affectation tools. Some embodiments of the present invention are designed to fill this need.

Further, all living cells, e.g. eukaryotic cells, may be electrically active. Heart cells grown outside a body in-vitro will all synchronize and beat together even though they are not part of a complete heart. Cells operate and communicate with other cells both electrically and chemically. Live cells can be affected by electric stimulation as in heart pacemakers. Cells also emit electrical frequencies and voltages that can be measured with tools like electroencephalographs and electrocardiographs. Neural tissues generate oscillatory activity in many ways, driven either by mechanisms localized within individual neurons or by interactions between neurons. An electric eel can discharge electrical bursts up to 600 volts at lethal currents.

Many diseases may be essentially a condition wherein operational processes of at least one cell may behave in an “aberrant” manner relative to the behavior of healthy cells of the same type. This behavior can take the form of shape variations, genetic variations, electrical charge and waveform expression variations, molecular variations, and/or chemical variations.

The pharmaceutical industry produces specific drugs to attempt to direct a disease condition back towards a “normal” condition, i.e. healthy state. Almost universally, there are undesired side effects in this approach. A primary objective of the present invention is to provide a device, system, and/or method for selectively redirecting the viable processes of a diseased live cell and/or a diseased live cell group back towards a “normal” condition. Another objective of the present invention is to cause cells that may be eluding the attention of the immune system to change their electrical “identification” processes so they may be recognized by the immune system.

The following reference list may represent relatively recent prior art disclosures that may be relevant to some embodiments of the present invention:

U.S. Pat. No. 7,993,906—Closed-loop electrical stimulation system for cell cultures
U.S. Pat. No. 8,728,139—System and method for energy delivery to a tissue using an electrode array
U.S. Pat. No. 8,718,756—Optimizing characteristics of an electric field to increase the field's effect on proliferating cells
U.S. Pat. No. 8,706,261—Treating a tumor or the like with electric fields at different frequencies
U.S. Pat. No. 8,447,396—Treating bacteria with electric fields
U.S. Pat. No. 8,406,870—Treating cancer using electromagnetic fields in combination with other treatment regimens
U.S. Pat. No. 7,890,183—Treating parasites with electric fields
U.S. Pat. No. 7,722,606—Device and method for destruction of cancer cells
U.S. Pat. No. 7,519,420—Apparatus for selectively destroying dividing cells

The present invention is the result of many years of confidential research into electrical and electromagnetic stimulation of cells to promote accelerated healing, the potential reactivation of stem cell activity, hard and soft tissue regeneration, as well as immune system stimulation to combat cancer, heart disease, and general autoimmune dysfunction.

No prior art live cell electrical signal and molecular signature sensing and affecting system exists that is configured to capture detailed electrical signal data from live cells, reduce that data to subsets and electrical “words”, create a “dictionary” of live cell electrical word definitions, and then transform live cell processes using electrical words drawn from said electrical signal dictionary. The present invention provides this capability.

### SUMMARY OF THE INVENTION

The present invention may integrate wideband frequency generators, transducers, sensors, frequency analyzers, wavelength meters, light power meters, cameras, computers, and computer software to discover “bioactive frequencies” through computer image analysis of the effects of electrical and electromagnetic frequencies on living tissues to promote accelerated healing.

The present invention may also incorporate a database with fields and lookup tables populated from tissue and cell reactions data derived from real time image capture and event tracking software that locks on to cell behavior changes in response to electrical and electromagnetic frequencies applied to the cells. These datasets are continually updated as effects are observed and quantified, and the electrical and electromagnetic frequencies are automatically modified and augmented to accelerate beneficial changes. For example, if a certain frequency clearly speeds up cell division during a frequency sweep, that frequency may be locked in, and harmonics of that frequency at different points in the electromagnetic spectrum may be added with the goal of further accelerating beneficial changes in cell behavior while reducing the electrical power required.

In the present invention, frequencies ranging from DC to x-rays—comprising sub nanometer resolution monchromated light—are applied to live cell samples for both observation and affectation. Both non contact electromagnetic field generating transducers in close proximity to a live cell sample, and direct electrical application to live cell samples through electrodes fitted to microscope slides are used to apply the frequencies to the cells. The electrode fitted microscope slides may incorporate multiple electrical contact zones to apply voltage and current at various frequencies to live cell samples, read the changes in the impedance of the samples, and read the characteristics of the frequency waves (sine, square, etc.) passing through the samples. There may be a minimum of three contact points per zone—positive in, positive out, and ground—with all zones able to be wired in parallel or dealt with separately. The voltage levels may be quite high—up to 400 volts—but the current may be small—microamp to milliamp levels. Each zone may be 20 by 20 microns or smaller. This may be achieved with clear conductive overlays like cell phone touch screen flexible conductive films affixed to microscope slides.

Impedance matching may be necessary to correctly apply the required frequency and energy level outputs to induce desired results since tissue density and impedance changes with body depth and any electrical or electromagnetic frequency applied. Tissue simulators are used in ultrasound transducer calibration and may also be used in the present invention to set benchmarks for signal amplitude output ratios and impedance matching to account for the differences in signal penetration between a fully functional living body and a sample containing just a few living cells.

In some embodiments of the present invention, a high gain amplifier may be coupled to a live cell direct contact electrical signal-sensing array. Said array may be in turn coupled to an amplifier and an analog to digital converter and/or a computer to detect, record, and digitize the natural electrical signals, pulses, and waveforms that a given cell type may use to communicate with its neighbors, the immune system, and the organism in which it is operating, as well as the electrical signals, pulses, and waveforms that may define its particular natural operational electrical signature.

Specific parts of the recorded electrical signals that are determined to be relevant to specific cellular functional processes may be extracted from the recordings as discreet waveforms—or “words”—in waveform editing software, and converted into a file format that may then be loaded into an arbitrary waveform generator. The resulting cellular signal words may be played back through an amplifier that is coupled to either an indirect radiating electrical transducer or coupled to direct electrical contacts applied to the cells in vitro, the organism, the body, or the cells within the body to affect the cells' electrical behavior.

In the present invention, electrical signals may be captured and analyzed from both healthy and diseased cells from the same or different cell types both in-vitro and in-vivo. Subsets (words) of the recordings may be extracted and “played back” to the cells whose behavior is to be modified. For example, the behavior change “catalyzing signal” may be extracted from a diseased cell or a cancer cell exhibiting a “death” signal in response to a chemical or electrical stimulus. The application of this signal may induce a death effect in the remaining cancerous or otherwise diseased cells. It is well known that all cells have a “death” trigger signal (apoptosis) inherent in their operational mechanics that kicks in when the body or that particular tissue is no longer capable of continuing to operate in a viable state. However, the exact electrical structure of these signals is not known in prior art. The present invention may reveal these signals and the structure of these signals.

In another example of the use of the present invention, an electrical signal derived from a healthy cell may be applied to a diseased cell to trigger it to return to a healthy state.

In another example of the use of the present invention, a signal from a healthy cell that indicates its type—such as liver signal—may be applied to a cancer tumor made up of liver cells that have metastasized to a lung and are “hiding” by putting out signals that direct the immune system to ignore it. The application of certain electrical “words” may induce the tumor cell group to announce its existence to the immune system—thereby activating the body's own defenses against the tumor—causing remission of the cancer condition.

A primary objective of the present invention may be to expand a catalog of frequencies used in the electro medical tool industry by providing a research toolset to observe and affect live cells and live tissue samples in real time with high resolution, high contrast live cell imaging analysis under both direct electrical contact and non-contact electromagnetic wave stimulation. The present invention may provide better frequency choices to enhance the healing processes in virtually every cell type in human and other animal bodies.

An objective of the present invention may be to define electrical and electromagnetic frequencies that may promote the enhanced uptake of beneficial drugs, vitamin and mineral compounds in a living organism.

Another primary objective of the present invention may be to identify and record specific electrical signals over time that may be inherent in healthy and/or diseased living cells, extract certain electrical signal subsets of said electrical signals, and then reapply those electrical signal subsets—or modified versions of those electrical signal subsets—to cells in a precise manner to selectively change specific functional electrical characteristics of living cell or tissue. The device, system, and/or method may be used to direct diseased or aberrant cells operating in the body to return to a normal state—or in the case of cancer cells for example—to cause the cancer cells to be recognized by the immune system and subsequently destroyed by the immune system.

Another objective of the present invention is to create a “dictionary” of live cell communications, with definitions associated with the “words” extracted from the electrical vocabulary used by all cell types—both healthy and diseased—in a human or other animal body.

The above, and other objects, features and advantages of the present apparatus will become apparent from the following description read in conjunction with the accompanying drawings, in which like reference numerals designate the same elements. A more complete understanding of the present invention, as well as further features and advantages, will be obtained by reference to the following detailed description and drawings. Preferred embodiments of the present invention will be described in the claims.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry may not be depicted in order to provide a clear view of the various embodiments of the invention

FIG. 1 is a schematic key component diagram illustrating an electro medical tool optimization system per the present invention.

FIG. 2 is a slide per the present invention fitted with conductive traces contacting a cell sample.

FIG. 3 is a microscope head per the present invention fitted with transmission and pickup coils, as well as a conductive trace fitted slide.

FIG. 3A is a microscope head per the present invention fitted with a waveguide, as well as a conductive trace fitted slide.

FIG. 4 is a cell sample having been affected by an electromagnetic frequency per the present invention.

FIG. 5 is a detail of a conductive trace pattern that may be applied to microscope slide per the present invention.

FIG. 6 is a series of simulated accelerated cell changes with cell perimeter and detail mapping per the present invention.

FIG. 6A is a series of simulated accelerated cell changes with cell perimeter and detail mapping as well as cell uptake of a chemical compound per the present invention.

FIG. 7 is a schematic diagram of the process of the preferred embodiment of the present invention.

FIG. 8 is a schematic diagram of the process of the preferred embodiment of the present invention comprising molecular analysis functionality.

FIG. 9 is a chart recording of electrical signal outputs from a cell culture with PC3 confluent cancer cells.

FIG. 10 is a chart recording of electrical signal outputs from PC3 confluent cancer cells stressed with the introduction of an alcohol solution in highly diluted but toxic concentrations, indicating a waveform subset to be loaded into an arbitrary waveform generator.

FIG. 11 is a chart recording of electrical signal outputs from a PC3 confluent cancer cell culture that was treated with the waveform shown in FIG. 10 for three minutes resulting in significantly reduced electrical amplitude output and observable electrical waveform output changes.

### DETAILED DESCRIPTION OF THE INVENTION

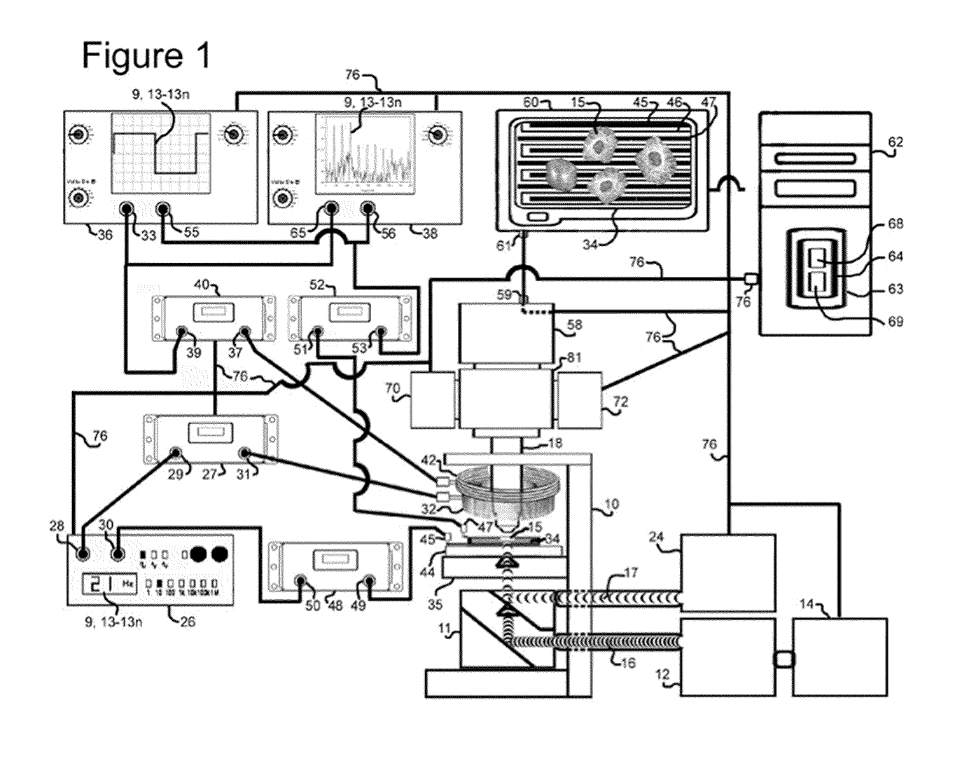

A preferred embodiment of an electro medical tool optimization system in accordance with the present invention per the system flow chart schematic as shown in FIG. 1 may provide the capability to detect a specific live cell 15 anywhere in a live cell sample 34 containing multiple cells, and subsequently apply electrical or electromagnetic signals to a cell sample 34 and modify the behavior of any specific cell 15. Said electrical or electromagnetic signals may be drawn from a frequency range comprising DC through x-rays. The present invention is designed to effect live cells, observe and collect data on said effects, and use said data to optimize said effects to catalyze a specific cell function modification or neutralization.

The preferred embodiment of an electro medical tool optimization system in accordance with the present invention is shown in FIG. 1 through FIG. 6a, and all said Figures with duplicate, expanded, or detailed renderings of specific elements will share the same numbers for said elements. In FIG. 1 specifically, cell sample 34 and cell 15 are represented simultaneously below lens 18 and as part of a screen image in video display 60.

As presented in FIG. 1, the present invention may include a microscope 10, which may be configured with a monochromator 12 capable of splitting a white light source 14 into single nanometer or sub nanometer wavelengths 16 that may range from 200 to 1100 nanometers, but said wavelengths 16 in practice may only be limited by the state of the art in monochromator 12 technology. Monochromator 12 may output any of said wavelengths 16 through any cell 15 in a cell sample 34 and on through a lens 18 of microscope 10.

Monochromator 12 may be operated manually or it may be electrically coupled to computer 62 that is configured with software program 63. Software program 63 may be configured to direct monochromator 12 to provide desired specific wavelengths 16.

Alternatively, software program 63 may be configured to control a variable color light source 24 that is electrically coupled to a computer 62 and capable of outputting any one of millions or billions of colors 17, said colors 17 in practice may only be limited by the state of the art in computer software and variable color light source 24 technology. Source 24 may also be configured to output said colors 17 through any cell 15 in a cell sample 34 and on through a lens 18 of microscope 10. Source 24 may be a video projector, an RGB color laser array, a super continuum laser, or any other variable color emitting light source that derives its color output commands manually by a user or from a software program 63 on a computer 62. A dual beam combiner 11 may also be used to combine and aim the light outputs from monochromator 12 and source 24 through any cell 15.

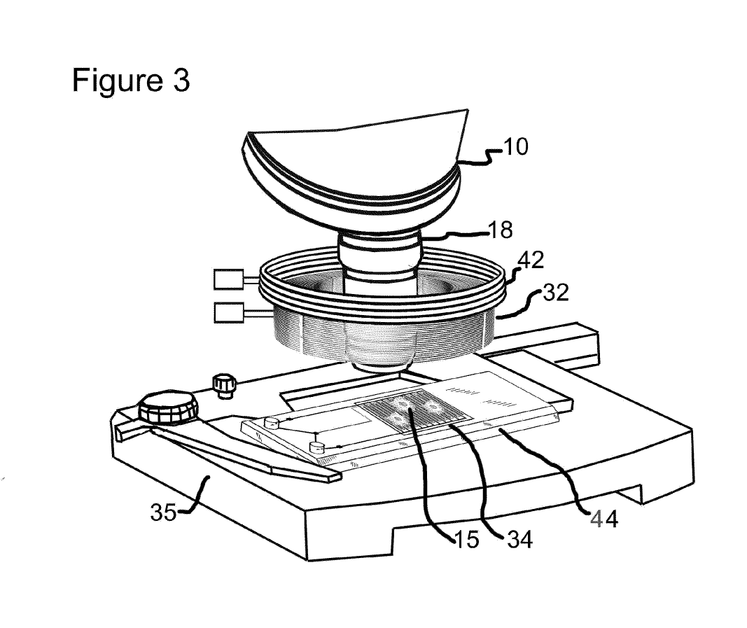

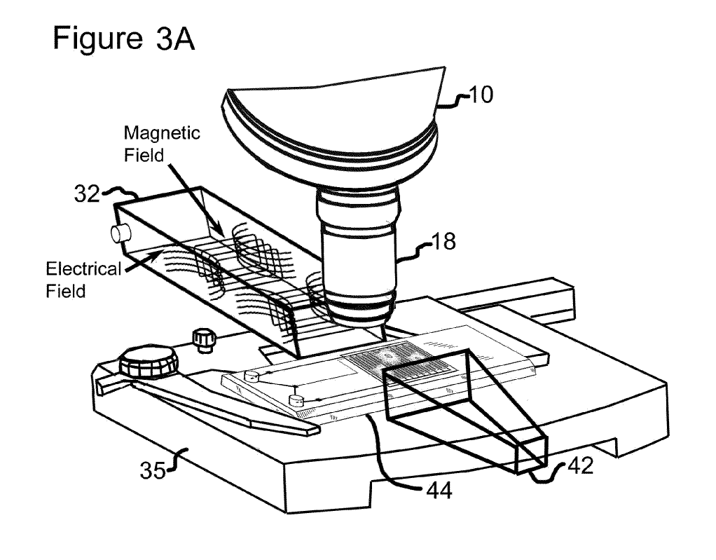

A waveform generator 26 with at least two channels of signal outputs 28 and 30, may be electrically coupled to a wideband power amplifier 27 input 29 through waveform generator output 28. Amplifier 27 output 31 may be electrically coupled to an electromagnetic field output transducer 32 which may be mechanically mounted to microscope 10 to surround a microscope lens 18 to induce an electromagnetic field into any cell 15 in cell sample 34 placed on a microscope stage 35. In certain cases, waveform generator 26 may be also be electrically coupled directly to electromagnetic field output transducer 32 through waveform generator output 28 by bypassing amplifier 27. Electromagnetic field output transducer 32 may be single transducer or a plurality of transducers configured to generate an electromagnetic field in response to any waveform 9 provided by waveform generator 26. In certain frequency ranges electromagnetic field output transducer 32 may be a waveguide as shown in FIG. 3A.

Waveform generator 26 may output any standard function generator waveform 9 comprising but not limited to sine, square, ramp, pulse, noise, DC, as well as a user defined waveform 9 with sweep functionality, variable duty cycle, variable amplitude, and variable frequency range from DC up through the AC electromagnetic spectrum. The Anritsu MG3690C with frequency extender options can cover near DC to 500 gigahertz in one box. For the purposes of the present invention said waveform generator 26 frequency range in practice may only be limited by the current state of the art.

Waveform Generator 26 is also connected to computer 62 through instrument interface 76 to allow data transfer and functional control by software program 63. All components presented herein are connected to computer 62 through instrument interface 76 for bidirectional data transfer. These data couplings between computer 62 and all other components described in the present invention may be USB, RS232, Firewire, GPIB, or any other industry standard instrumentation data interface. The electrical signal couplings between all components may be BNC cables or any other industry standard.

Power amplifier 27 should have frequency response equal to waveform generator 26. waveform generator 26 may also have an input 25 which is electrically coupled to computer 62. Electromagnetic field output transducer 32 should have frequency response equal to waveform generator 26.

The input 37 of a wideband amplifier 40 may be electrically coupled to an electromagnetic pickup transducer 42 which may be mechanically mounted concentrically to transducer 32 on microscope 10 such that it can detect any electromagnetic waves that may be applied to the proximity of any cell 15 in cell sample 34 by electromagnetic transducer 32. Amplifier 40 ideally may eliminate any impedance mismatches in pickup transducer 42 as any waveform 9 may be applied to transducer 32 from waveform generator 26 through power amplifier 27. Amplifier 40 may have the same frequency response as amplifier 27.

Electromagnetic pickup transducer 42 may be a single transducer or a plurality of transducers configured to sense an electromagnetic field in response to any waveform 9 provided by waveform generator 26. In certain frequency ranges electromagnetic pickup transducer 42 may be a waveguide as shown in FIG. 3A.

An oscilloscope 36 input channel 33 and a spectrum analyzer 38 input channel 65 may be electrically coupled in parallel to the output 39 of amplifier 40. Oscilloscope 36 and spectrum analyzer 38 may have the same frequency response as waveform generator 26 and may be used to insure that any waveform 9 applied to transducer 32 are in fact reaching cell sample 34. Oscilloscope 36 and spectrum analyzer 38 may also be directly electrically connected to transducer 42 and in certain configurations of the present invention by bypassing amplifier 40. Additionally, oscilloscope 36 and spectrum analyzer 38 functions may be incorporated into a single spectrophotometer device.

In the preferred embodiment of the present invention in FIG. 1, waveform generator 26, wideband power amplifier 27, microscope stage 35, wideband amplifier 40, oscilloscope 36, spectrum analyzer 38, wideband power amplifier 48, wideband amplifier 52, camera 58, wavelength meter 70, and optical power meter 72 may all be electrically coupled to computer 62 through industry standard instrumentation interface 76 to allow functional control by software program 63.

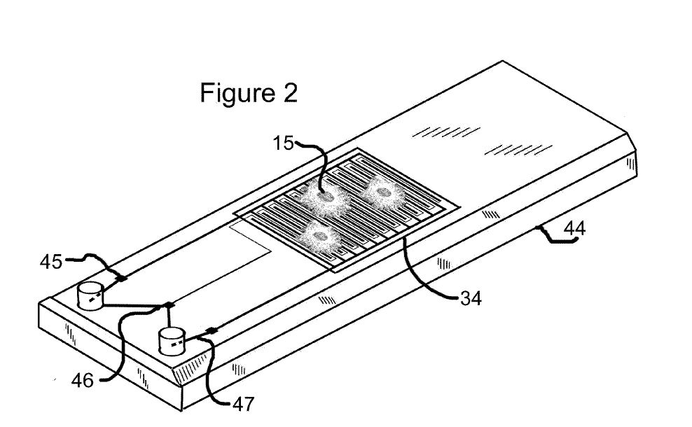

As detailed in FIG. 2, cell sample 34 may also be contained on an electrically conductive microscope slide 44. Slide 44 may be configured with a minimum of three electrically conductive traces 45, 46, and 47 configured to make electrical contact with any cell 15 in cell sample 34.

Referring again to FIG. 1, slide 44 and traces 45, 46, and 47 are shown expanded on display 60 to further clarify their configuration. Trace 46 may be the ground connection for input trace 45 and output trace 47. Slide 44 input trace 45 may be electrically connected to wideband power amplifier 48 output 49. Input 50 on amplifier 48 may be electrically connected to output 30 of waveform generator 26. Power amplifier 48 should have frequency response equal to waveform generator 26.

Slide 44 output 47 may be electrically connected to input 51 of a wideband amplifier 52. Amplifier 52 output 53 may be electrically coupled in parallel to oscilloscope 36 second input channel 55 and spectrum analyzer 38 second input channel 56. Amplifier 52 ideally may eliminate impedance mismatches in any cell 15 in a cell sample 34 as any waveform 9 may be applied to traces 45 and 47 from waveform generator 26 through power amplifier 48. Amplifier 52 should have the same frequency response as waveform generator 26.

Camera 58 may be mounted on a beam splitter 81 on said microscope 10 so as to view cell sample 34 through lens 18. A video display 60 input 61 may be coupled to video output 59 of camera 58 to allow a user to monitor any effect of waveform generator 26 waveform 9 on any cell 15 in a cell sample 34. Additionally, video output 59 may be also electrically coupled to computer 62 through instrument interface 76. Software program 63 may be configured to track and map any cell 15 in cell sample 34 and populate database 64 with cell 15 size and cell mechanics data derived from image data provided by camera 58.

Baseline cell behavior information 68 may be included in database 64. Baseline cell behavior information 68 may include typical cell size for a given cell type, rate of mitosis, molecular pathway openings and closings relative to certain chemical compounds, etc. Software program 63 may be configured to detect any cellular behavior alteration 69 from camera 58. Cellular behavior alteration 69 may include any cell 15 behavior deviation from a baseline cell behavior information 68, comprising such changes as size and shape. Baseline cell behavior information 68 may be refreshed with respect to initial cell 15 size data at the start of every experiment.

Wavelength meter 70 and optical power meter 72 may also be installed on beam splitter 81 on microscope 10 to log the spectral information of a cell sample 34 before and after application of said waveforms 9 from waveform generator 26. Wavelength meter 70 and optical power meter 72 may be electrically coupled to computer 62 through instrument interface 76 and information they provide may continually populate relevant fields in database 64. Software program 63 may be configured to modify the waveforms 9 in response to any cellular behavior alteration 69 occurring in response to any waveform 9, light wavelength 16, or light color 17. Wavelength meter 70 and optical power meter 72 may have the same frequency response as monochromator 12 and source 24. Wavelength meter 70 and optical power meter 72 may also be a single spectrometer device incorporating the functions of both.

As camera 58 image data updates database 64, cellular behavior alteration 69 data may enable software program 63 to track hundreds of cells in a cell sample 34 simultaneously.

In database 64, each cell 15 location within cell sample 34 on slide 44 may be represented in the x/y/z axes relative to a “zero” point on a three dimensional environment model mapped to the observable area of a microscope slide 44 in database 64 at a resolution of 0.2 microns, or a resolution only limited by current state of the art in optical lens technology. This type of “object of interest” microscopic targeting and tracking software is now available from an array of software providers.

Each cell 15 initial size may be logged with the same resolution of 0.2 microns, or a resolution only limited by current state of the art in optical lens technology.

Any cellular behavior alteration 69 data may be logged and updated in real time continually updating field data in database 64 as managed by software program 63.

An array of statistical outputs from cellular behavior alteration 69 data may include:

a. real time updated position information of any cell 15 in environmental model.
b. acceleration/deceleration of any cell 15 in real time and over time.
c. expansion/contraction of any cell 15 in real time and over time.
d. ambient fluid flow into/out of any cell 15 in real time and over time.

A user of the present invention may initially set waveform generator 26 to sweep a waveform 9 from DC up to the frequency limits of waveform generator 26 at a particular sweep rate not to exceed the image and data acquisition limits of camera 58. When any cellular behavior alteration 69 occurs, the waveform 9 being output at that moment may be locked in by a user or software program 63 and the signal amplitude may be raised or lowered or the pulse width or duty cycle may be altered. Waveform generator 26 may then be directed by a user or software program 63 to add a harmonic 13 of root waveform 9 to waveform 9. Additional harmonics from 13 p to 13n, as well as wave shape and amplitude alterations may also be tested until no more cellular behavior alteration 69 occurs.

A user may also manually choose to lock-in or sweep through the light wavelength 16 outputs of monochromator 12 and variable color source 24 colors 17. Software program 63 may also be configured to sweep through and lock in any wavelength 16 or colors 17 of light, or combinations of the light outputs of monochromator 12 and variable color source 24 at a particular sweep rate not to exceed the image and data acquisition limits of camera 58.

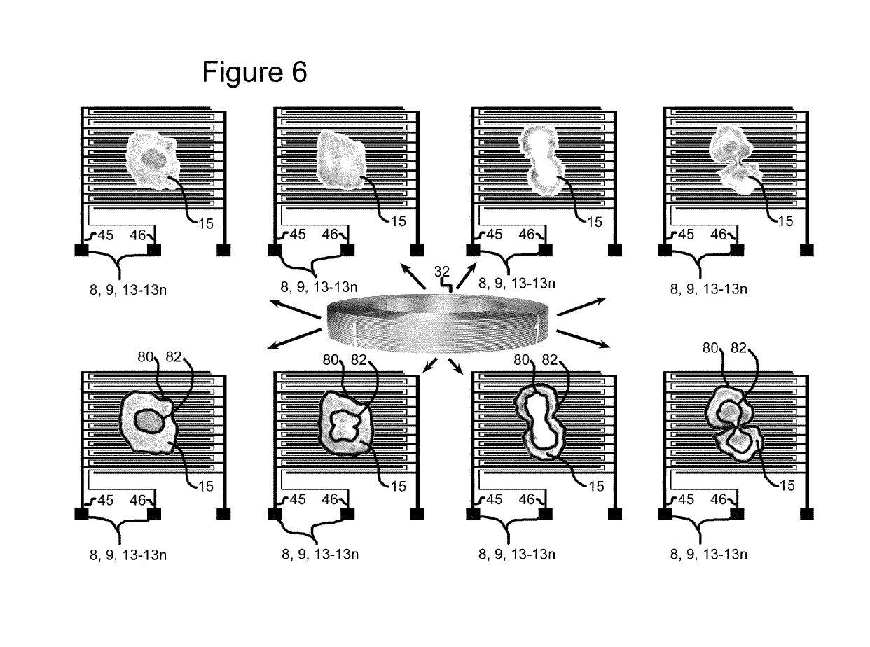

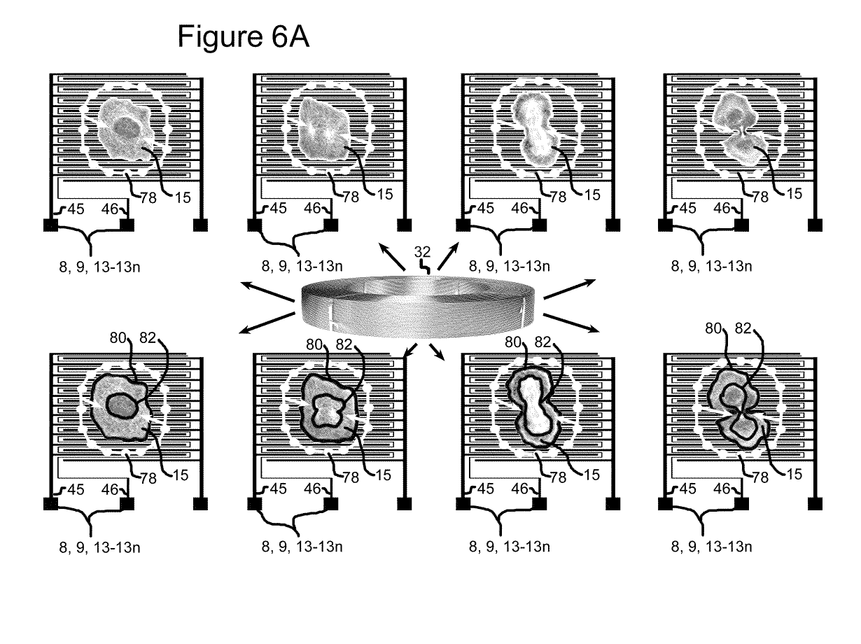

Those frequency waves that are most absorbed by a cell 15 may be considered for the purposes of the present invention as indicated in FIG. 6, resonant frequencies 8 of a cell 15. Said resonant frequencies 8 are a subset of any available waveform 9 and must initially be identified through laboratory experimentation, and are then integrated within database 64 as lookup tables. “Overdriving” the amplitude of said resonant frequencies 8 with respect to a base rate of frequency absorption of a cell 15, said rate data contained in database 64, may affect the electrical conductivity and the chemical and mechanical conditions of a cell 15. These resonant frequencies 8 may then be manipulated and augmented by changes in pulse rate, amplitude, and wave shape, as well as the addition of frequency inversions, harmonics, and dissonances of said resonant frequencies 8 by software program 63 through computer 62 and waveform generator 26. It may be the manipulation of these resonant frequencies 8, combined with other waveforms 9 and one or more of harmonic 13 through 13n combinations that catalyze a cellular behavior alteration 69.

Resonant frequencies 8, combined with other waveforms 9 and harmonic 13 through 13n may be applied to catalyze destruction of a cancerous cell 15 mechanics—initially by altering a single specific structure or behavior within a cell 15, and then outputting and altering additional applied waveforms 9 and harmonic 13 through 13n combinations, which may then propagate state changes in other cell 15 structures and mechanics like a domino effect—possibly allowing an immune system to recognize a cancer cell 15 within a cell sample 34 as an invader and dispatch white blood cells to destroy it. For example, if a molecule of a given component may be comprised of ten atomic elements arranged in a particular way, modifying the polarity of the third most abundant atomic element in the molecule may have such a catalyzing effect on a cell 15 mechanics.

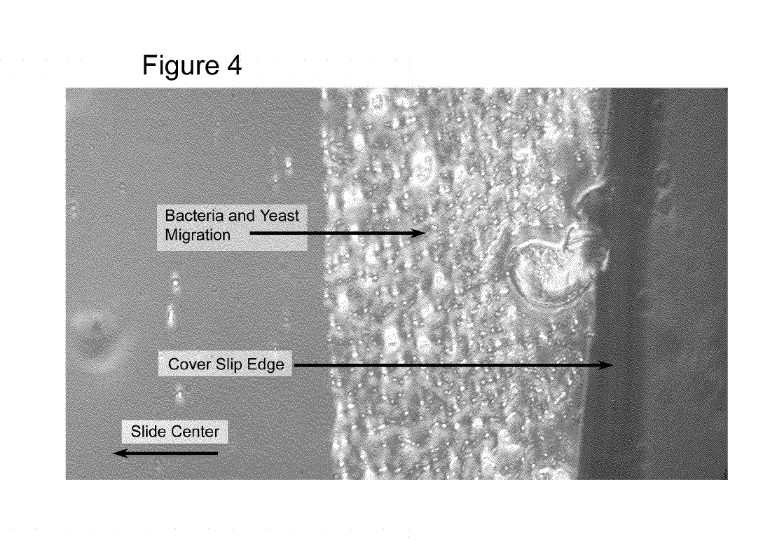

Referring now to FIG. 4 which is a photograph from a laboratory experiment using the present invention, sine wave frequencies of 727 hertz were applied to a cell sample 34 consisting of residue from a wine tank. Said cell sample 34 contained many bacteria and yeast cell 15 examples in large numbers. After a few minutes it was observed that almost a cell 15 elements migrated to the cover slip edges of slide 44—leaving the middle of the cover slip area almost empty. This is a simple example of electromagnetic effects on organisms at the microscopic level.

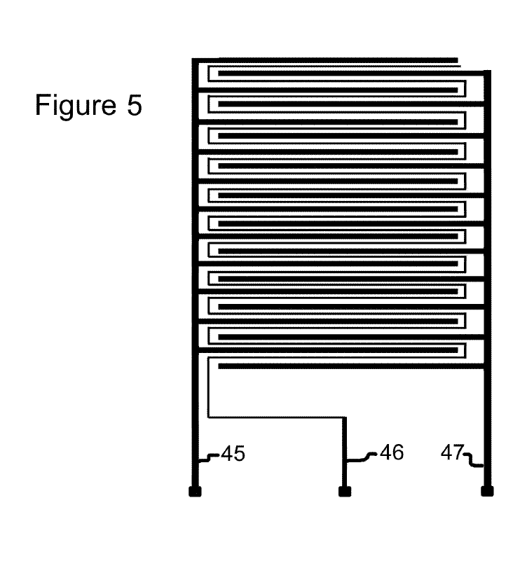

FIG. 5 is one possible trace detail per the present invention that may be rendered on a clear conductive film that may be applied to a glass or polycarbonate microscope slide. Said clear conductive film traces 45, 46, and 47 may be printed or separated by conductivity-neutralized areas.

Referring now to FIG. 6, a resonant frequency 8, individually or combined with a waveform 9 and a harmonic 13 through 13n combinations may be applied to a cell 15 through traces 45 and 46 and/or electromagnetic transducer 32 to stimulate a cellular behavior alteration 69 in the form of simulated accelerated mitosis as seen in the series of cell 15 changes illustrated in the top row. In the bottom row of FIG. 6, said cellular behavior alteration 69 may be mapped (by software program 63 in response to image data derived from camera 58 as detailed in FIG. 1) as black outline 80 around the perimeter of cell 15 and black outline 82 around the nucleus 83 of a cell 15. The black arrows indicate electromagnetic waves radiating from electromagnetic transducer 32.

Referring now to FIG. 6A, a catalytic bioavailable media 78 which may include a drug, a vitamin, or a mineral compound, in conjunction with a resonant frequency 8, individually or combined with a waveform 9 and a harmonic 13 through 13n combinations may be applied to a cell 15 through traces 45 and 46 and/or electromagnetic transducer 32 to stimulate a cellular behavior alteration 69 in the form of simulated accelerated mitosis as seen in the series of cell 15 changes illustrated in the top row. In the bottom row of FIG. 6A, said cellular behavior alteration 69 may be mapped (by software program 63 in response to image data derived from camera 58 as detailed in FIG. 1) as black outline 80 around the perimeter of cell 15 and black outline 82 around the nucleus 83 of a cell 15. The black arrows indicate electromagnetic waves radiating from electromagnetic transducer 32.

Many of the elements and software capabilities included in the present invention are available in various industries and disciplines so they are not detailed herein beyond the description presented. However, the present invention is a unique and novel system and apparatus combination that incorporates methods, features, and components not found in any other single apparatus or toolset.

There may be three primary aspects of all biological systems—mechanical, chemical, and electrical. The present invention may focus on the electrical signals, pulses, waveforms, and mechanisms that cells use to communicate to their neighbors, the brain, and the body. Cells' electrical language may include both “sentences” and “words.” Electrically, cells may speak relatively slowly. Much of cellular electrical dialog may happen at similar speeds to low frequency brain waves, and some cellular electrical dialog may be much slower. Cellular electrical language may appear similar on many levels. While this inventor was preparing prototypes of the present invention, several neuroscientists were invited to review the raw waveform data extracted from various unstimulated live cell types. All of the neuroscientists pointed to parts of the waveform recordings as signals consistent with brain neuron activity, cardiac cellular activity, and/or muscular activity. All of these neuroscientists were shocked to learn that the signals were derived from small groups of live cells that were not neurons, cardiac cells, nor muscle cells.

The present invention may record the electrical “language” of healthy and diseased live cells. The details and differences—the “words”—of that language may be compared between healthy and diseased cells, and between varying cell types. Certain key words from the language may be extracted, reconfigured as to file type, and reapplied to re-educate badly behaving—or diseased—cells through repeated application of said words to modify the electrical condition of live cells toward a desired behavioral (viable) condition.

In some preferred embodiments of the present invention, a cell sample 34 may be grown on an electrode array 44, or grown in a standard cell culture petri dish and may be electrically coupled to an overhead, drop down, variation of said electrode array 44. The electrodes comprising said array 44 may be configured to provide multiple points of contact to create a pathway for electrical signals emanating from, and being applied to, a live cell 15 in a cell sample 34. Array 44 may be configured as an interdigitated differential electrical contact with two positive leads as trace 45 and trace 47, and a common ground 46. Array 44 may also be used as a simple positive trace 45 and a negative connection 46 for single sided signal output and input. Pluralities of electrical traces in array 44 may provide greater live cell 15 or cell sample 34 surface area contact that may provide additional signal channel inputs or better signal to noise ratio.

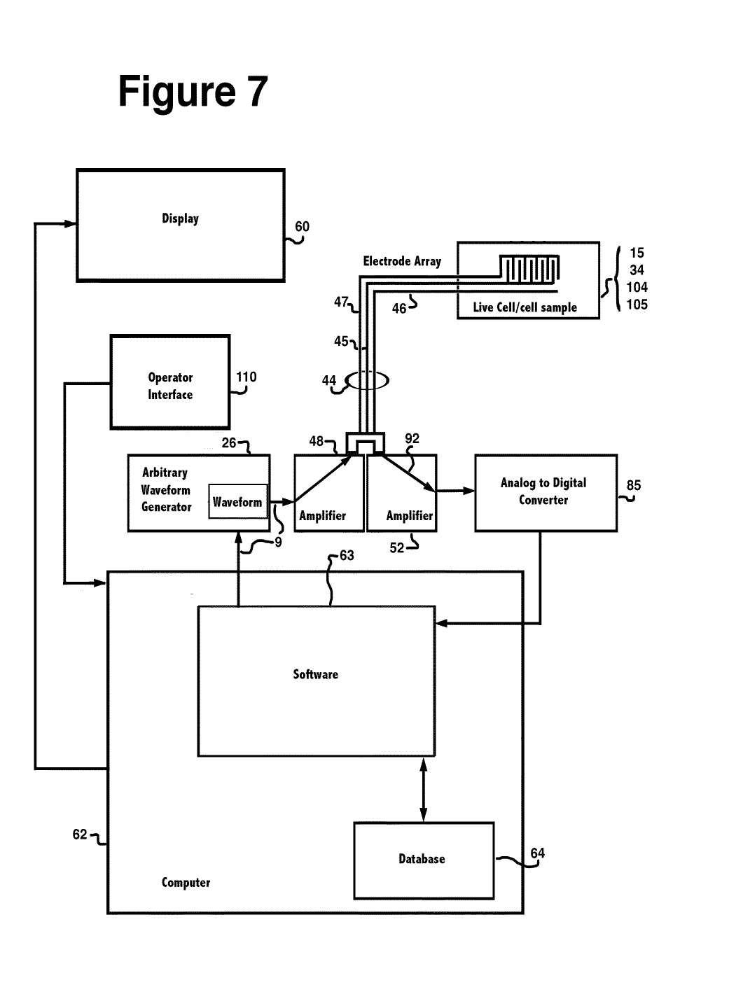

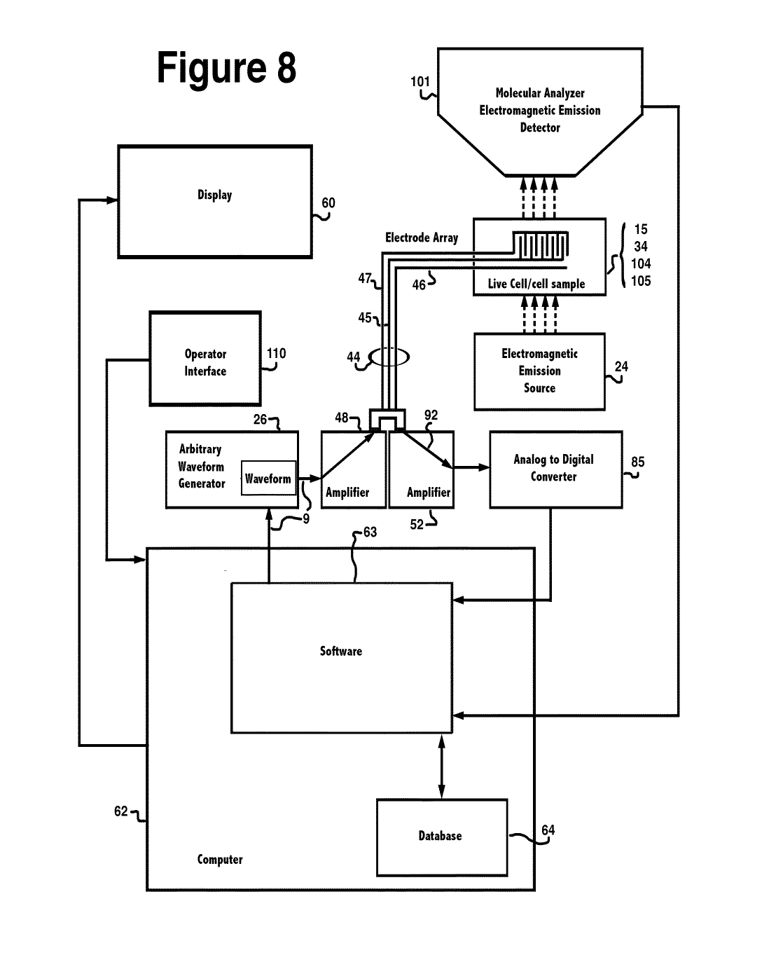

In some preferred embodiments, array 44 as represented in FIG. 7 and FIG. 8 may be presented as an exemplary single interdigitated differential electrode configuration. Electrically conductive trace 45 and electrically conductive trace 47 in electrical signal array 44 may be used as differential positive signal inputs, and trace 46 is used as a ground connection. Array 44 may be electrically coupled to signal input amplifier 52 and signal output amplifier 48. Array 44 may be substantially equivalent to electrical conductive slide 44, or may be adhered to a different material substrate or even as discrete unmounted electrodes applied from any direction as long as contact is made with a live cell 15, or a cell sample 34, a live cell 105, or a cell sample 104.

In some preferred embodiments operational elements in the present invention may electrically interconnect through industry standard instrumentation interface 76. Said interface 76 may not be indicated with a numeral in all of the drawing Figures herein, but is considered to be part of any preferred embodiment where it would be effective.

In a preferred embodiment as depicted in FIG. 1, cell 15 and/or cell sample 34 may provide electrical signal information to amplifier 52, then said cell 15 and/or cell sample 34 may be replaced by cell 105 and/or cell sample 104, wherein cell 105 and/or cell sample 104 may then be stimulated by the electrical signal output process of some embodiments of the present invention, wherein array 44 may be coupled to amplifier 48.

Either trace 45 or 47 may be used as a signal positive in a non-differential configuration, but differential inputs may be inherently lower in noise than single ended inputs. Amplifier 52 may be a high variable gain, low noise amplifier such as a Grass JP511. However, any analog or analog to digital amplifier package that provides representative electrical signals characteristics of a cell 15 or cell sample 34 may be used. The Texas Instruments ADS family combines multi-channel analog preamplifiers with analog to digital conversion, and may function effectively. An analog amplifier may be used in some preferred embodiments because of subtleties that may be detectable in said cell 15 or cell sample 34. For example, and without limiting the scope of the present invention, some such subtleties may include cellular reaction to electrical and/or chemical stimuli.

An AD Instruments Powerlab SP analog to digital converter 85 may be used in some preferred embodiments to convert the output from amplifier 52 to at least one plurality of electrical signals 92 derived from cell 15 or cell sample 34 so computer 62 may record said signals 92. Said signals 92 may be representative of a baseline cell behavior information 68 being a first viable state of a cell sample 34 or a live cell 15.

Any analog to digital converter 85 may be used that may be capable of correctly representing signals 92. In some preferred embodiments of the present invention signals 92 derived from a group of cells in sample 34 may more accurately represent electrical intercellular communication in or out of a living body than deriving signals 92 from just one cell 15.

At least one electrical signal “word” waveform 9 may be extracted from said signals 92 by at least one operator interface 110 electrically coupled to computer 62, or by software program 63 operationally installed on computer 62. Said at least one waveform 9 may be applied to at least one second live cell 105 or a second cell sample 104 to alter the state of viability from a cell baseline behavior 68 to a cell altered behavior 69, or to an otherwise healthy or normal state. Said at least one first cell 15 and said cell sample 34, and said at least one second cell 105 and said second cell sample 104, may be similar or entirely different cell types.

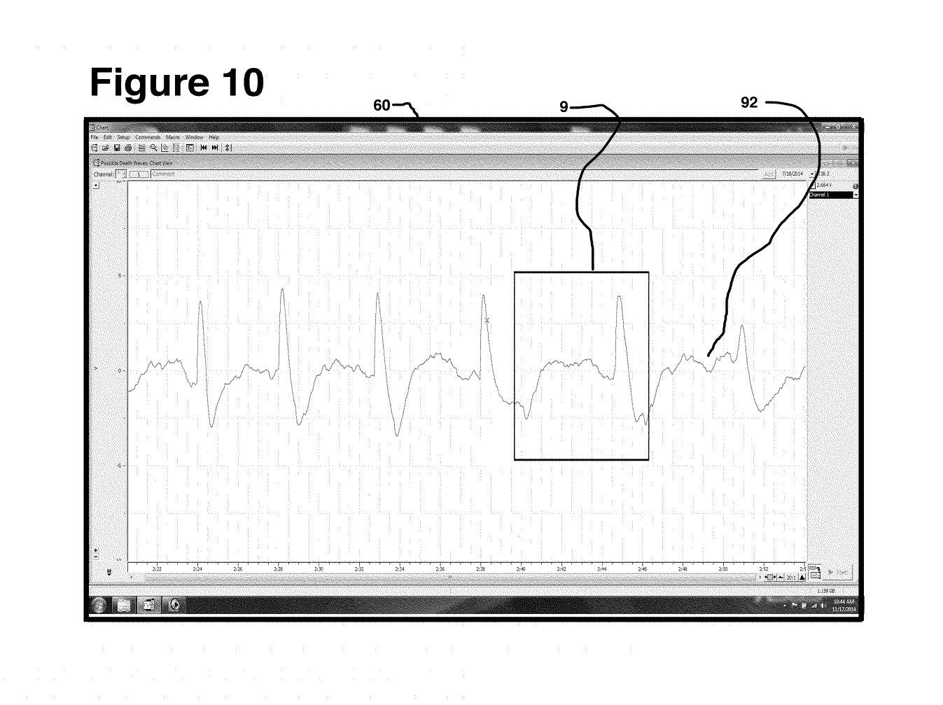

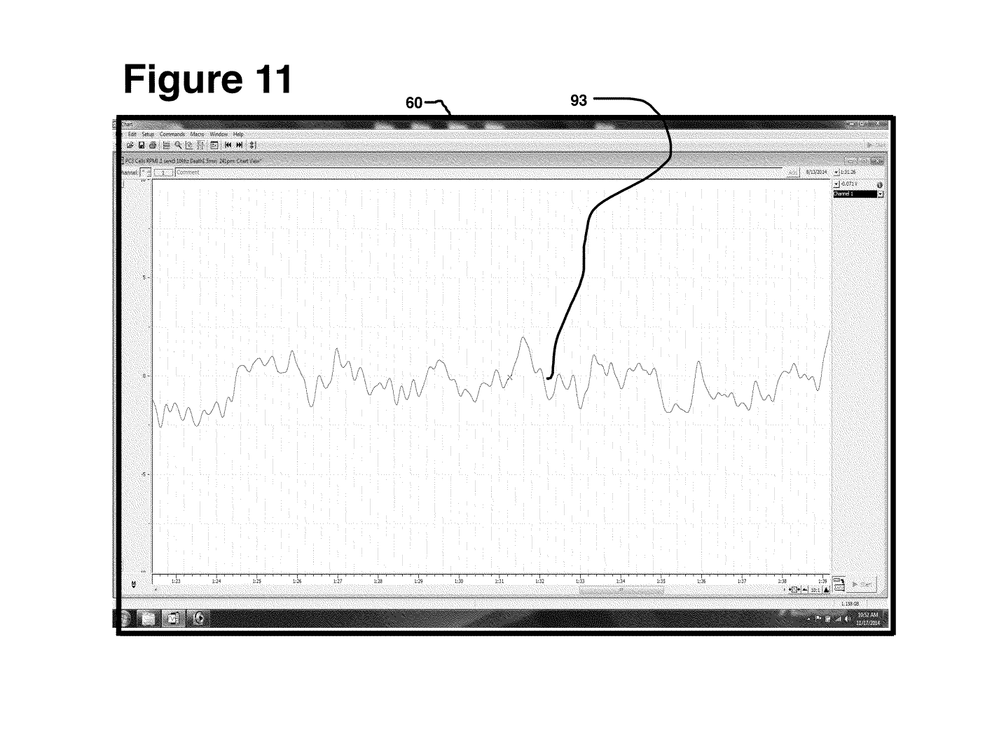

In some embodiments, analog to digital converter 85 may capture signals 92 as amplitudes, voltages, and pulses over time as a digital file that may be recorded and processed by software program 63 to provide a subset of said signals 92 as at least one waveform 9. Waveform 9 may then be uploaded to waveform generator 26 through software program 63. Said software program 63 may incorporate file editing and conversion functions that may be substantially equivalent to those in a program such as “Easywave” available from Siglent, who may manufacture a version of arbitrary waveform generator 26. Said software program 63 may also incorporate file conversion functions that may be substantially equivalent to those in the program “Chart” by AD Instruments, the maker of the Powerlab SP. Said Chart software may output waveform recordings as .wav files, .csv files, and the like. Other software such as Excel, Audigy, or Cool edit pro may also be used to convert .wav files into a waveform 9 may be uploaded into waveform generator 26. The format for said waveform 9 may be a .csv file, an excel file, or any other file format that may be uploaded to an arbitrary waveform generator 26. Any signals 92, or said waveform 9 may be also be loaded into database 64 such that software program 63 may be configured to direct computer 62 to output an appropriate waveform 9 as depicted in FIG. 10, through amplifier 48 and array 44, as depicted in FIG. 7 and FIG. 8, to alter the state of viability from a cell baseline behavior 68 to a cell altered behavior 69 in said at least one second live cell 105 or said second cell sample 104. Said cell altered behavior 69 may be represented by a second waveform signal 93 as depicted in FIG. 11. Said cell altered behavior 69 second waveform signal 93 may include altered cellular division, a change in immune system response, a change in molecular structure, entering a state of apoptosis, a change in metabolism, a change in DNA or RNA replication, a change in transcription, a change in translation, a change in cell signaling, a change in cell surface activity, a change in cell surface permeability, reactivation of stem cell functionality in adult differentiated cells, alteration of electrical conductivity or alteration of electrical conductivity in an ambient medium surrounding said at least one biological cell, and altered uptake of drugs, vitamin or mineral compounds by said at least one biological cell, or a state of apoptosis being triggered in said at least one second live cell 104 or said second live cell sample 104.

Just as a pacemaker may alter the rhythms of a heart with subtle electrical signals, so may a waveform 9 may alter the electrical characteristics of a cell 105, or a cell sample 104 toward a desired result. When a waveform 9 may be applied to a cell 105, or a cell sample 104, array 44 may be rewired to serve as an electrical signal output device wherein trace 45 and trace 47 may be used as parallel positive outputs and trace 46 may be used as a ground connection from amplifier 48. Either trace 45 or trace 47 may used alone, but using the pair provides a larger surface area of electrical conductivity. Further, in certain embodiments of the present invention, amplifier 48 and amplifier 52 may be the same unit, simply switching input and output connections through interface 76 to array 44.

In some preferred embodiments of the present invention, an electrical signal waveform 9 introduced into array 44 by waveform generator 26 through amplifier 48, and applied to a cell 105, or a cell sample 104, may include direct current, direct current pulses or alternating current waveforms of any shape and frequency derived from a cell 15, cell sample 34, or the subsonic, acoustic, or electromagnetic spectrum. Array 44 electrode traces may be substantially equivalent to those on electrical conductive slide array 44 or may be adhered to a different material substrate or even as discrete unmounted electrodes.

As noted above, oscilloscope 36, spectrum analyzer 38, wavelength meter 70, and/or optical power meter 72 may also be configured as a single spectrometer device incorporating the functions of either or all, to provide the function of detecting electromagnetic emission molecular signature data in a live cell 15, cell sample 34, live cell 105, or cell sample 104. In some embodiments, light source 24 may provide the required electromagnetic energy emission to excite molecular shifts, i.e. light source 24 may operate as an electromagnetic energy emission source. In some embodiments, camera 58 may provide the function of detecting electromagnetic emission molecular signature data in a live cell 15, cell sample 34, live cell 105, and/or cell sample 104, depending on the sensor such as an MCT, microbolometer, or thermopile chosen for said camera 58, wherein said camera 58 may also function as molecular analyzer 101.

Referring to FIG. 8, in some embodiments a molecular analyzer 101 may operate as an electromagnetic emission detector. In some embodiments, molecular analyzer 101 may comprise one or more of the functions of wavelength meter 70, optical power meter 72, camera 58, oscilloscope 36, spectrum analyzer 38, and the like. Molecular analyzer 101 may be configured to directly view a live cell 15, cell sample 34, live cell 105, and/or cell sample 104 in a manner that does not incorporate a microscope 10.

Light source 24 as indicated above may provide any wavelength in the acoustic or electromagnetic spectrum, and as configured in some embodiments of the present invention, may be configured as an infrared energy emitter, a single wavelength laser, a quantum cascade laser, a supercontinuum laser, or any other source of electromagnetic waves able to excite molecular and atomic bonds so they may be sensed by said molecular analyzer 101. Said molecular analyzer 101 may be a Raman spectrometer, an infrared absorbance spectrometer, or any other detector able to sense molecular data in response to electromagnetic waves emissions.

Some embodiments of the present invention may be intended to take advantage of improvement in the disclosed components as said improvements become available. Dispersive infrared, FT-IR infrared, X-Ray, and Raman adapters are available for microscopes, and as stand alone tools, so no further detail on these components is required.

Analyzer 101 may be electrically coupled to computer 62 such that the absorbed, reflected and transmitted details of any cell 15, cell 105, cell sample 34, or cell sample 104, as sensed by molecular analyzer 101 are defined by software program 63 as molecular signatures of any cell 15 or cell sample 34 and entered into appropriate field and columns in database 64.

The benefit of incorporating real time molecular signature analysis may be that it provides an enhanced additional cell 15, cell 105, or cell sample 34 or cell sample 104 viability feedback mechanism to optimize a desired electrical effect on cell 105, or cell sample 104.

Using video display 60, computer 62, software program 63, database 64, and operator interface 110, a user may manually derive a waveform 9 and load said waveform 9 into waveform generator 26 to create a desired change in electrical viability in a cell 15, cell 105, cell sample 34, or cell sample 104. The present invention may also operate automatically as disclosed herein and as detailed above.

Some embodiments, of the present invention as presented in FIG. 7 may incorporate a configuration of array 44 as an EEG, EMG, or EKG (ECG) placement on a live animal or human subject to record said signals 92 from said live animal or human subject as brainwave or heartbeat signals 92 and apply said signals 92 or a waveform 9 subset of said signals 92 to a live cell 105 or a cell sample 104 to develop data relevant to the automatic and autonomic nervous system and cell behavior relationship to alter the state of viability from a cell baseline behavior 68 to a cell altered behavior 69.

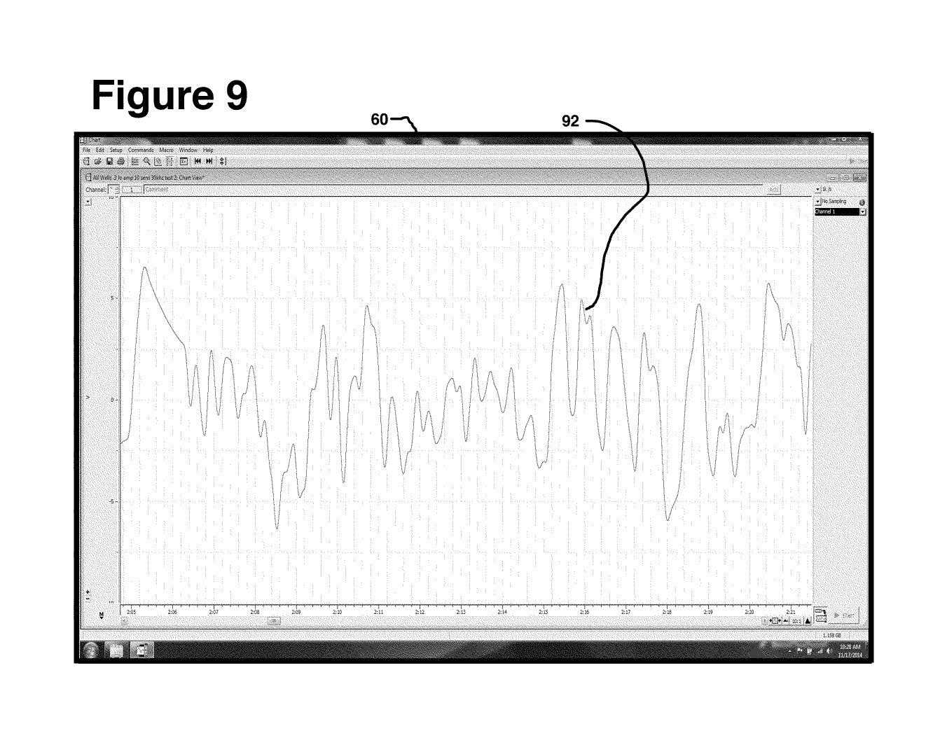

As presented in FIG. 11, a representative cell viability electrical waveform signal 93 indicates a dramatically lowered electrical signal output from a cell sample 105 after the application of a waveform 9 through array 44 after said waveform 9 was extracted from a signal 92 recording over time represented in FIG. 10, that resulted from the application of a toxic chemical stimulus to said first cell sample 34 signal 92 also shown in part in FIG. 9 prior to said toxic chemical introduction, demonstrating that a preferred embodiment of the present invention may alter the state of viability from a cell baseline behavior 68 to a cell altered behavior 69. In the upper left corner of FIG. 9 (landscape orientation) a “sens” factor of thirty was used to acquire the unstimulated cell 15 baseline behavior 68, and the same location on FIG. 11 it can be seen that the “sens” setting was at five. That means it took approximately 6 times the gain setting to detect any electrical output from second cell sample 104 following application of waveform 9 extracted from said signals 92 depicted in FIG. 10.

While the invention has been particularly shown and described with reference to preferred embodiments thereof, it will be understood by those skilled in the art that various changes in form and details may be made without departing from the spirit and scope of the invention.

I claim:

1. A live cell viability modification system comprising:
at least one first live cell;
at least one electrically conductive array;
at least one amplifier electrically coupled to said at least one electrically conductive array,
said at least one electrically conductive array in contact with at said least one first live cell in a first viable state;
an analog to digital converter electrically coupled to said amplifier to digitize and record a plurality of first electrical signals sensed from said at least one first live cell through said at least one electrically conductive array;
a computer configured to extract at least one second electrical signal from said plurality of first electrical signals;
at least one arbitrary waveform generator electrically coupled to said at least one amplifier, wherein said at least one arbitrary waveform generator is configured to generate said at least one second electrical signal;
wherein said at least one second electrical signal is generated from said arbitrary waveform generator through said at least one amplifier and through said electrically conductive array to at least one second live cell.

2. A live cell viability modification system according to claim 1, wherein said at least one second live cell was sensed behaving in the first viable state prior to exposure of said at least one second electrical signal, wherein upon exposure to said at least one second electrical signal said at least one second live cell is sensed behaving in a second viable state.

3. A live cell viability modification system according to claim 1, wherein said at least one second live cell is sensed behaving in a second viable state prior to exposure of said at least one second electrical signal, wherein upon exposure to said at least one second electrical signal said at least one second live cell is sensed behaving in the first viable state.

4. A live cell viability modification system according to claim 1, wherein said at least one second viable state is selected from one or more of the group consisting of: altered cellular division, a change in immune system response, a change in molecular structure, entering a state of apoptosis, a change in metabolism, a change in DNA or RNA replication, a change in transcription, a change in translation, a change in cell signaling, a change in cell surface activity, a change in cell surface permeability, reactivation of stem cell functionality in adult differentiated cells, alteration of electrical conductivity or alteration of electrical conductivity in an ambient medium surrounding said at least one biological cell, altered uptake of drugs, vitamin or mineral compounds by said at least one biological cell, and said at least one second live cell is triggered into initiating apoptosis.

5. A live cell viability modification system according to claim 1 comprising at least one operator interface electrically coupled to said computer to allow a user to extract at least one second electrical signal from said plurality of first electrical signals.

6. A live cell viability modification system according to claim 1 comprising at least one electromagnetic energy emission source configured to focus electromagnetic emissions toward said at least one live cell.

7. A live cell viability modification system according to claim 1 comprising at least one electromagnetic energy emission detector configured to determine the absorption, transmission, or reflectance of at least one electromagnetic energy emission source interacting with said at least one live cell.

8. A live cell viability modification system according to claim 1 wherein said at least one computer incorporates at least one database populated with said at least one first cell viable state data that is compared to said at least one second live cell viable state data wherein said comparison is used to extract said second electrical signal.

9. A live cell viability modification system according to claim 1 comprising brainwave and heartbeat acquisition, analysis, and amplification.

10. A live cell viability modification system according to claim 1 that includes a “dictionary” of live cell electrical signals, with at least one cell functionality definition associated with said at least one second electrical signal.

11. A live cell viability modification system according to claim 1, wherein said at least one second electrical signal is further modified with harmonics, frequency, phase, or amplitude shifts.

12. A method for modifying live cell viability, comprising the steps of:
placing at least one first live cell in physical contact with at least one electrically conductive array, wherein said at least one first live cell is in a first viable state;
sensing a plurality of first electrical signals from said at least one first live cell by at least one analog to digital converter, wherein said analog to digital converter is electrically coupled to said at least one electrically conductive array; wherein said analog to digital converter is configured to digitize and record the plurality of first electrical signals;
extracting at least one second electrical signal from said plurality of first electrical signals; wherein the step of extracting is performed with the use of a computer;
loading said at least one second electrical signal into at least one arbitrary waveform generator that is electrically coupled to said computer;
placing at least one second live cell in physical contact with at least one electrically conductive array;
applying said at least one second electrical signal to said at least one second live cell; wherein the step of applying is performed by said at least one arbitrary waveform generator; wherein said at least one arbitrary waveform generator is electrically coupled to said at least one electrically conductive array.

13. A method for modifying live cell viability according to claim 12, further comprising a step of sensing a first viable state of said at least one second live cell prior to the step of applying said at least one second electrical signal to said at least one second live cell.

14. A method for modifying live cell viability according to claim 12, further comprising a step of receiving at least one command from a user engaging at least one operator interface, wherein said at least one operator interface is electrically coupled to said computer, and said at least one command is a command to extract at least one second electrical signal from said plurality of first electrical signals.

15. A method for modifying live cell viability according to claim 12, further comprising a step of directing focused electromagnetic emissions toward said at least one first or second live cell; wherein said focused electromagnetic emissions are emitted from at least one electromagnetic energy emission source.

16. A method for modifying live cell viability according to claim 12, further comprising the step of detecting absorption, transmission, and/or reflectance of directed focused electromagnetic emissions from said at least one first or second live cell, wherein the detecting is performed by at least one electromagnetic energy emission detector.

17. A method for modifying live cell viability according to claim 12, further comprising a step of extracting said second electrical signal by incorporating use of at least one database populated with said at least one first cell viable state data that is compared to said at least one second live cell viable state data; wherein the extracting is performed by said computer pursuant to instruction in software.

18. A method for modifying live cell viability according to claim 12, further comprising a step of applying electrical signals associated with brainwaves and/or heartbeats to said second live cell.

19. A method for modifying live cell viability according to claim 12, further comprising a step of building a dictionary of live cell electrical words, wherein said dictionary comprises at least one live cell electrical word entry, wherein said at least one live cell electrical word entry comprises at least one detected electrical signal associated with said at least one first or second live cell, and wherein for each at least one detected electrical signal is at least one cell functionality definition observed with said at least one detected electrical signal.

20. A method for modifying live cell viability according to claim 12, further comprising a step of modifying said at least one second electrical signal; wherein the modifying is a change in one or more of harmonics, frequency, phase, and/or amplitude shifts of said at least one second electrical signal.

21. A method for modifying live cell viability according to claim 12, wherein said at least one second viable state is selected from one or more of the group consisting of: altered cellular division, a change in immune system response, a change in molecular structure, entering a state of apoptosis, a change in metabolism, a change in DNA or RNA replication, a change in transcription, a change in translation, a change in cell signaling, a change in cell surface activity, a change in cell surface permeability, reactivation of stem cell functionality in adult differentiated cells, alteration of electrical conductivity or alteration of electrical conductivity in an ambient medium surrounding said at least one biological cell, altered uptake of drugs, vitamin or mineral compounds by said at least one biological cell, and triggering apoptosis in said at least one second live cell.
